(19) United States
(12) Patent Application Publication
(10) Pub. No.: US 2026/0308657 A1
(43) Pub. Date: Oct. 8, 2026

(54) A SYSTEM FOR IDENTIFYING MICROBIAL GROWTH

(71) Applicant: MICROPLATE DX LTD., Glasgow, Scotland (GB)

(72) Inventors: Stuart Hannah, Glasgow, Scotland (GB); Damion Corrigan, Glasgow, Scotland (GB); Paul Hoskisson, Glasgow, Scotland (GB); Michael Malecha, Glasgow, Scotland (GB); Heather Ralston, Glasgow, Scotland (GB)

(21) Appl. No.: 19/484,534

(22) PCT Filed: May 16, 2024
(86) PCT No.: PCT/EP2024/063543

§ 371 Date: Nov. 14, 2025

(30) Foreign Application Priority Data
May 16, 2023 (GB) 2307287.9

(51) Int. Cl. C12Q 1/18; C12M 1/32; C12M 1/34; C12M 1/36; C12Q 1/02; G01N 27/327

(52) U.S. Cl. CPC C12Q 1/18; C12M 23/12; C12M 41/36; C12M 41/46; C12M 41/48; C12Q 1/025; G01N 27/327

(57) ABSTRACT

Disclosed herein is a system for identifying microbial growth. The system comprises a cartridge for receiving a sample that may comprise a pathogen, the cartridge comprising an array of wells, each well comprising an electrochemical transducer. The system also comprises a processor configured to interrogate each of the electrochemical transducers, wherein interrogation of the electrochemical transducers comprises, for each well: generating a first data series by repeatedly measuring a first dependent variable; and generating a second data series by repeatedly measuring either the first, or a second dependent variable. The processor is further configured to determine, based on the first and second data series from each well, whether there has been microbial growth in any of the wells.

This disclosure relates to determining whether there has been microbial growth, and in particular relates to a system and computer-implemented method for determining whether there has been microbial growth in a well of an array of wells. Optionally, methods and systems of the present disclosure may be used for the purpose of performing antimicrobial (e.g. antibiotic) agent susceptibility testing.

### BACKGROUND

Methods of antimicrobial susceptibility testing (AST) provide information regarding both the susceptibility and the potential resistance of one or more pathogens to antimicrobial agents. AST methods help healthcare professionals make decisions on the most suitable drug for a particular patient, and help assure susceptibility of drugs for particular infections.

Currently, AST methods are based on cell growth, and require incubation over relatively long periods of time. For example, known methods focus on the application of traditional growth of bacterial cultures overnight using microbroth dilution (BMD), disk diffusion (DD) or similar standard methods. These known methods rely on an overnight culture to be cultivated from the original sample, which introduces a significant time delay between a decision to perform AST and obtaining the results. Typically, this time delay is on the order of two days, but it can take much longer. It typically takes at least 18 hours, for example, from setting up a growth test before a cell count may be attempted, and this would typically be prior to any AST being performed, since it may take 18 h simply to perform the initial overnight culture step.

This approach is sub-optimal, as prompt administration of appropriate antibiotics is associated with improved outcomes for patients. Known AST techniques, while typically providing accurate results which are useful for healthcare professionals, do not enable rapid decisions to be made at the point of care. It will therefore be appreciated that the delays introduced by known AST techniques can negatively impact the level of care that healthcare professionals can provide to patients. Further, while waiting for AST results, the healthcare provider may administer a sub-optimal antibiotic, for example a broad spectrum antibiotic. This can be problematic, since the overuse or misuse of antibiotic drugs may contribute to antimicrobial resistance, leading antibiotic agents to become less effective over time.

It is known to use an electrode system comprising two or more electrodes to test the electrical response of a substance which is suitable for electrical conductance and which is capable of microbial growth. Using this system and methodology, a difference in electrical response from the substance between a first time and a second, later time may be indicative of microbial growth having occurred between the first and second time. While this system and method is advantageous for many reasons, it remains desirable to reduce the “time to positive” still further, and also to go beyond a simple identification of microbial growth.

In summary, existing AST methods introduce a significant delay before an optimal antibiotic can be administered, and this not only negatively impacts patient care but also contributes to the global problem of antimicrobial resistance.

The present invention seeks to address these and other disadvantages encountered in the prior art by providing an improved system for antibiotic susceptibility testing.

### SUMMARY

According to an aspect, there is provided a system for identifying microbial growth, the system comprising a cartridge for receiving a sample that may comprise a pathogen. The cartridge comprises an array of wells, each well comprising an electrochemical transducer. The system comprises a processor configured to interrogate each of the electrochemical transducers, wherein interrogation of the electrochemical transducers comprises, for each well: generating a first data series by repeatedly measuring a first dependent variable; and generating a second data series by repeatedly measuring either the first, or a second dependent variable. The processor is further configured to determine, based on the first and second data series from each well, whether there has been microbial growth in any of the wells.

According to another aspect, there is provided a computer-implemented method suitable for identifying microbial growth. The method is suitable for use with a system comprising a cartridge for receiving a sample that may comprise a pathogen, the cartridge comprising an array of wells, each well comprising an electrochemical transducer. The method comprises interrogating each of the electrochemical transducers, wherein interrogation of the electrochemical transducers comprises, for each well: generating a first data series by repeatedly measuring a first dependent variable; and generating a second data series by repeatedly measuring either the first, or a second dependent variable. The method further comprises determining, based on the first and second data series from each well, whether there has been microbial growth in any of the wells.

According to another aspect, there is provided a computer readable medium comprising computer-executable instructions which, when executed by a processor, cause the processor to perform a computer-implemented method suitable for identifying microbial growth. The method is suitable for use with a system comprising a cartridge for receiving a sample that may comprise a pathogen, the cartridge comprising an array of wells, each well comprising an electrochemical transducer. The method comprises interrogating each of the electrochemical transducers, wherein interrogation of the electrochemical transducers comprises, for each well: generating a first data series by repeatedly measuring a first dependent variable; and generating a second data series by repeatedly measuring either the first, or a second dependent variable. The method further comprises determining, based on the first and second data series from each well, whether there has been microbial growth in any of the wells.

### FIGURES

Specific embodiments are now described, by way of example only, with reference to the drawings, in which:

FIG. 1 depicts a system according to the present disclosure;

FIG. 2 depicts a method according to the present disclosure;

FIG. 3 depicts a method according to the present disclosure;

FIG. 4 is a graph depicting the output of one or more numerical models according to the present disclosure, and in particular shows estimated cell concentration over time in both an inhibited and an uninhibited well;

FIG. 5 is a graph depicting the output of one or more numerical models according to the present disclosure, and in particular shows estimated cell concentrations over time for a plurality of subsets of wells, including a baseline subset, a control subset, and five inhibited subsets;

FIG. 6 is a graph depicting the output of one or more numerical models according to the present disclosure; and in particular shows the output of a model which has been tuned to generate results in a reduced time period;

FIG. 7 is a graph depicting the output according to a generalised linear model according to the present disclosure;

FIGS. 8A and 8B show a matrix multiplication technique which may be used in numerical models according to the present disclosure;

FIG. 9 is a graph depicting the output of a partial least squares model according to the present disclosure;

FIG. 10 is a graph depicting the output of a principal component regression model according to the present disclosure; and

FIG. 11 depicts a flowchart depicting a method according to the present disclosure.

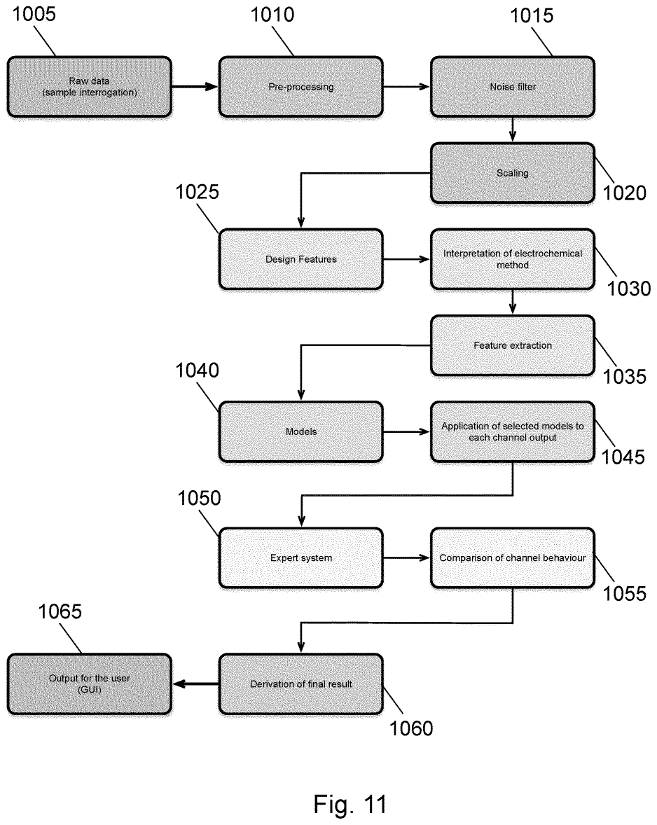

### DETAILED DESCRIPTION

In overview, and without limitation, the application discloses a system for identifying microbial growth, and which (optionally) is also able to perform antimicrobial susceptibility testing. The system comprises a cartridge for receiving a sample. The cartridge comprises an array of wells, each well comprising an electrochemical transducer. Some of the wells hold a first antibiotic agent, whereas some other wells on the cartridge do not hold the first antibiotic agent (control wells). In implementations in which the goal is not only to determine if a particular antibiotic is appropriate, but to determine which of multiple antibiotic agents is most effective, then the cartridge has multiple wells comprising each of the antibiotic agents in addition to the control wells.

Once a sample has been introduced into each of the wells, the electrochemical transducers in each well are interrogated. A number of scans are performed sequentially in each cell. A first variable is measured over time to generate a first data series. A second data series is generated by additionally measuring either the first, or a second dependent variable over time. Suitable dependent variables to measure during the electrochemical interrogation process include current, capacitance, impedance, a first derivative of the impedance, a second derivative of the impedance, phase, and voltage.

Therefore, to generate an example first data series, the frequency of a signal applied to each electrode may be controlled while monitoring changes in impedance. To generate an example second data series, the frequency of the signal applied to each electrode may be controlled while monitoring changes in capacitance. There may be additional data series generated during the interrogation process.

This multivariate and multivariable approach generates a wealth of data which can be used to determine whether a particular antibiotic agent is suitable for treating a bacteria comprised within the sample, or which of a number of candidate antibiotics is most suitable for treating the bacteria. It is this multivariate and multivariable approach which allows the time before a final determination is reached to be reduced from 2 days (as in traditional cell growth approaches) to 2 hours, or less.

Due to this significant reduction in testing time, methods of the present application are extremely valuable as a decision support tool to be utilised by healthcare professionals at the point of care. The methods allow a sample to be interrogated in real time. Bacterial growth can be established as early as 30 min into the test, with a further 30 min to distinguish if present bacteria are susceptible to the antibiotic. The presently disclosed system therefore enables doctors and other healthcare professionals to access vital diagnostic information, so they can prescribe the right drug at the right time.

While the present disclosure focuses primarily on a use case of AST, the skilled person will appreciate that the present methods are suitable for other use cases, for example biohazard monitoring for quality control in drink manufacture, and water quality monitoring.

### System

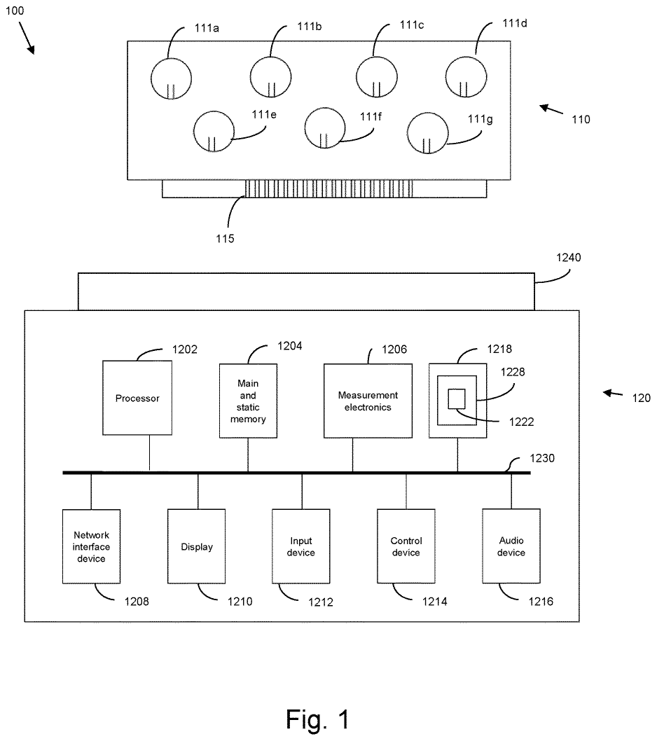

FIG. 1 depicts a system 100 according to the present disclosure. The system 100 comprises a cartridge 110 and a controller, which may be described as a control unit, 120.

The cartridge 110 may be referred to as a test cartridge. Depending on the specific implementation, the cartridge 110 may have several purposes, including to securely contain a sample from a patient, for example a urine sample; to filter the sample for impurities, for example via a 100 μm filter; to contain a growth media and pH buffer and antibiotic agent(s); and to facilitate electrochemical interrogation of the sample via the control unit 120.

The cartridge 110 comprises a plurality of wells 111a-g. The wells 111a-g may be described as electrode-sensor wells. Each of the wells 111a-g comprises a transducer, in particular an electrochemical transducer. The electrochemical transducers allow electrochemical interrogation of a sample by an array of electrodes. Each electrode-sensor well comprises an independent transducer, which in turn comprises (not depicted in FIG. 1) a working electrode (WE), a counter electrode (CE), and a reference electrode (RE). Each electrode-sensor well 111a-g is physically separated from other cells (i.e. there is no electrode-sensor well 111 situated downstream from another electrode-sensor well 111). While only seven wells 111a-g are depicted in the schematic depiction in FIG. 1, depending on the implementation it should be appreciated that the cartridge 110 may comprise an array of many more of such wells.

Each electrode well comprises its own gel layer. The gel composition for a particular well will depend on the usage to which the cartridge is being applied, and which subset or ‘configuration’ of wells the particular well falls into. For example, when the cartridge will be used for AST, there are three main cell configurations:
Blank cell or negative control (no antibiotic agent, but bacteriostatic agent or bacteria filtering is present)
Uninhibited growth cell (no antibiotic agent, no bacteriostatic agent or no bacteria filtering)
Inhibited growth cell (antibiotic agent present, no bacteriostatic agent or no bacteria filtering).

In the simplified implementation depicted in FIG. 1, for example, it might be that well 111a is a blank cell, in which there is no antibiotic agent present in the gel layer. A bacteriostatic or bactericidal agent is present. Signals and data series generated from this well will be used during pre-processing methods. Wells 111b,c may be uninhibited growth cells, in which the gel layer comprises no antibiotic agent, no bacteriostatic agent and no bacteria filtering means. These wells are control (more specifically: positive control) cells. The remaining wells 111d-g are inhibited growth wells. The gel layer of wells 111d,e may comprise a first antibiotic (AB) agent, and the gel layer of wells 111f,g may comprise a second, different AB agent.

It will be appreciated that test cartridges according to the present disclosure may be tailored to the particular pathogen, e.g. infection, they are configured to test for (e.g. urinary tract infections, respiratory tract infections, fungal infections etc.). For example, using the example described above, the first AB agent incorporated within wells 111d,e may be an agent typically suited for a number of fungal infections, whereas the second AB agent incorporated within the wells 111f,g may be another antibiotic agent particularly suited for a number of other fungal infections. Introducing a patient sample to each well of the cartridge and employing the methods of the present disclosure (described below), it is possible to determine which of the first and second AB agents is most suitable, i.e. optimal, for treating the fungal infection comprised within the patient sample.

Suitable antibiotics to be featured on the cartridge include:
Amoxicillin (AMX)
Ciprofloxacin (CIP)
Fosfomycin (FOS)
Nitrofurantoin (NIT)
Trimethoprim/Sulfamethoxazole (TRS).

The concentration levels of the antibiotics used on the cartridge may be any of:
Breakpoint concentration (for urine) for the antibiotic in question
One dilution down from breakpoint (half of breakpoint concentration)
One dilution up from breakpoint (double of breakpoint concentration).

Upon introduction of a patient sample (e.g. urine) into the cartridge 110, the sample is distributed through channels into each of the wells containing gel-modified electrode sensors. As soon as the sample reaches the gel present in each well 111a-g, bacterial growth may occur, which is monitored through electrochemical changes in parameters such as impedance in a manner which will be described in detail below. The presence of certain antibiotics may prevent this growth. At a high-level, the present methodologies may be used to indicates whether a pathogen, e.g. an infection, is present (i.e. is an antibiotic required), and if a pathogen is present, will select the optimum antibiotic candidates for subsequent clinical selection.

The device enables measurements of bacterial/fungal growth present in different types of clinical sample (blood, urine, sputum, cerebrospinal fluid) and a broad range of bacterial and fungal infections. The cartridges 110 of the present disclosure therefore allow a complete suite of antibiotics typically prescribed for a particular pathogen to be tested in the presence of one standard patient sample. For most sample types, there are no additional pre-processing steps required for the sample. In particular, for a UTI-configured cartridge, there are no pre-processing steps required for the urine sample.

Further, the device 100 comprises electronic communication means 115, which enables communication with the cartridge receiving means 1240. Signals can be passed between the cartridge 110 and the control unit 120 to enable the electrochemical transducers on the cartridge to interrogate the wells 111a-111g, and pass the resulting information back to control unit 120.

The system 100 also comprises a controller, in particular a control unit 120, which may take the form of a bench-top instrument. An advantage of the present system is that the control unit 120 is designed to sit on the bench at the point of care, and be positioned as close to the patients' bedside as possible. FIG. 1 depicts one specific implementation of a control unit 120 according to the present disclosure, but the skilled person will appreciate that a control unit according to the present disclosure may take many forms and that not all of the components shown in FIG. 1 are essential. Further, while only a single control unit 120 is illustrated, the term “controller” shall also be taken to include any collection of machines (e.g., computers) that individually or jointly execute a set (or multiple sets) of instructions to perform any one or more of the methodologies discussed herein.

Depending on the use case and specific implementation, the purpose of the control unit 120 is to house the cartridge 110, drive the test protocol (sample processing & interrogation), perform measurement(s), and provide an interface with the operator (for example a clinician).

The control unit 120 comprises cartridge receiving means 1240, which comprises at least an opening or aperture sized, positioned and otherwise configured for receiving the cartridge 110. The cartridge receiving means 1240 further comprises cartridge reading means configured to interact with the electronic communication means 115 on the cartridge.

The control unit 120 comprises one or more processors 1202. The processor(s) 1202 are configured to send control signals in order to interrogate the electrochemical transducers on the cartridge 110, as well as to receive and process signals received from those transducers, in a manner that will be described in detail below.

The one or more processors 1202 may comprise both a microcontroller (MC and a session border controller (SBC). This enables the software residing on the control unit to follow a split architecture of microcontroller (MC) and session border controller (SBC), in order to separate safety critical software components from non-safety critical ones. This structure has the added benefit of the non-safety critical software to be configurable to suit connectivity needs without impacting the development of the safety critical aspects.

The micro-controller handles safety critical software. The purpose of the micro-controller is to drive the basic functionality of the control unit 120 (mechanical, thermal and electronics parts). This includes:
Self-checks relating to the hardware components
Controlling the Analogue Front End (AFE)
Running data processing
Regulating temperature in the wells
Executing the processing logic (e.g. instructions 1222) for performing the algorithms, methods, operations and steps discussed herein.

The control unit 120 further comprises, a main memory and/or a static memory 1204. For example, a main memory may comprise read-only memory (ROM), flash memory, dynamic random access memory (DRAM) such as synchronous DRAM (SDRAM) or Rambus DRAM (RDRAM), etc. A static memory may comprise flash memory, static random access memory (SRAM), etc). The control unit 120 may further comprise a secondary memory (e.g., a data storage device 1218). The one or more processors 1202 communicate with each type of memory via a bus 1230.

The computing device 1200 may further include a network interface device 1208. The computing device 1200 also may include a video display unit 1210, an input device 1212 (e.g., a keyboard or touchscreen), a cursor control device 1214 (e.g., a mouse or touchscreen), and an audio device 1216 (e.g., a speaker) for providing feedback to an operator of the control unit 120. Components 1210, 1212, and 1214 may be combined to form a graphical user interface (GUI).

The data storage device 1218 may include one or more machine-readable storage media (or more specifically one or more transitory or non-transitory computer-readable storage media) 1228 on which is stored one or more sets of instructions 1222 embodying any one or more of the methodologies or functions described herein. The instructions 1222 may also reside, completely or at least partially, within the main memory 1204 and/or within the one or more processors 1202 during execution thereof by the control unit 120, the main memory 1204 and the processor 1202 also constituting computer-readable storage media. Accordingly, disclosed herein is a computer readable medium comprising computer-executable instructions which, when executed by a processor, cause the processor to perform any steps or features of the computer-implemented method disclosed herein.

The system 100 further comprises a potentiostat. The potentiostat of the system may be suitable for any of electrochemical impedance spectroscopy (EIS) measurements; Cyclic Voltammetry (CV); linear Sweep Voltammetry (LSV); Staircase Voltammetry (SCV); Normal Pulse Voltammetry (NPV); Differential Pulse Voltammetry (DPV); Square-Wave Voltammetry (SWV); and the like. As such, the potentiostat is capable of analysing, for example, electrical impedance as a function of test frequency, i.e. the potentiostat comprises an impedance analyser.

The potentiostat is capable of real-time data capture to generate one or more data series. Fluctuations in impedance indicate variations in microbial growth over time. As the population and metabolic activity of the microbes rise, the resistance of the substance to current flow, which depends on frequency (i.e., impedance), also increases. It is believed that the balance of charged particles within the first substance changes as the microbes proliferate and metabolize the components within it. Therefore, tracking changes in impedance over time provides insight into the rate of microbial growth.

To evaluate several antimicrobial agents, the present device/system can be utilized by assigning a distinct electrode system for each agent being tested. In doing so, the system 100 can be described as comprising a collection of devices, each containing a unique antimicrobial agent or a combination of agents, as well as one or more devices without any antimicrobial agent. These devices, present in each well, are each electronically linked to a potentiostat.

### Method

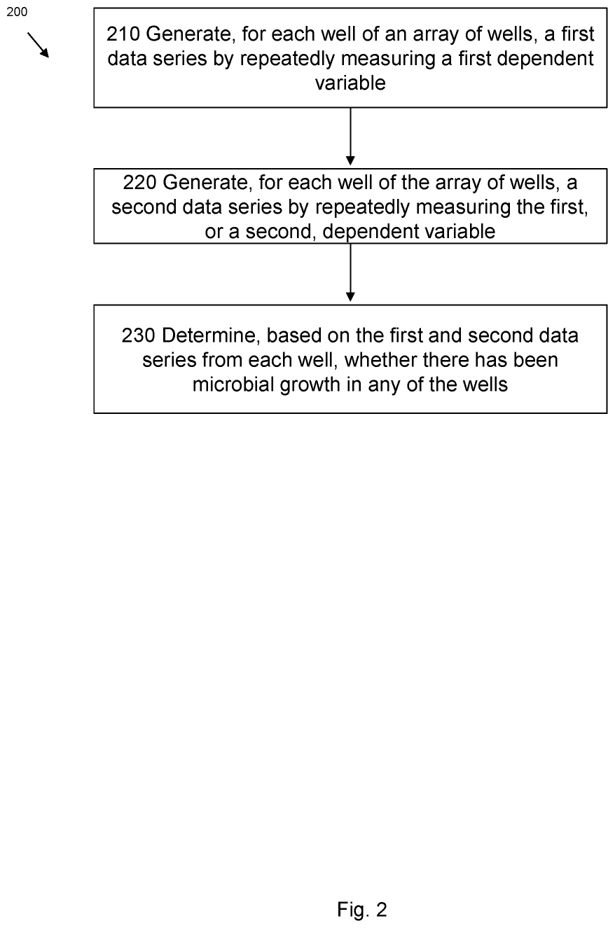

FIG. 2 is a flowchart depicting a method 200 according to the present application. The method is suitable for being performed by the one or more processors on the control unit described above in respect to FIG. 1. In particular, the method 200 may be performed when a cartridge 110 comprising a sample is inserted in the control unit 120. The sample may, or may not, comprise a pathogen. Before the method 200 is carried out, a sample is introduced to the cartridge. For example, the sample may be a urine sample from a patient who is suspected to have a UTI. The sample is introduced into each of the wells of the array of wells on the cartridge.

At the first block 210, the method 200 comprises generating, for each well of the array of wells, a first data series by repeatedly measuring a first dependent variable. At the second block 220, the method 200 comprises generating, for each well of the array of wells, a second data series by repeatedly measuring the first, or a second, dependent variable.

Each well of the array of wells on the cartridge comprises an electrochemical transducer, and generating the first and second data series is accomplished via a process of interrogating each of the wells, either sequentially on at the same time. For each data series generated as part of this interrogation process, the dependent or ‘measured’ variable may be any of current, capacitance, impedance, impedance′, impedance″, phase, and voltage. The data series may be generated by repeatedly measuring the relevant dependent variable while controlling an independent variable, such that the first data series is generated at block 210 by repeatedly measuring a first dependent variable while controlling a first independent variable, whereas the second data series may be generated by repeatedly measuring either the first, or the second dependent variable while controlling either the first, or a second, independent variable. The dependent variables may be any of current, capacitance, impedance, a first derivative of the impedance, a second derivative of the impedance, phase, and voltage. The first and second independent variables are selected from voltage, frequency, and time. This is shown in table 1 below:

| Independent | | | Dependent | |
|---|---|---|---|---|
| variable | Unit | Range | variable | Unit |
| Voltage | V | −0.1 to 0.75 | Current | μA |
| Frequency | Hz | 0.1 to 100000 | Capacitance | F |
| Frequency | Hz | 0.1 to 100000 | Current | μA |
| Frequency | Hz | 0.1 to 100000 | Impedance | Ω |
| Frequency | Hz | 0.1 to 100000 | Impedance ′ | Ω |
| Frequency | Hz | 0.1 to 100000 | Impedance ″ | Ω |
| Frequency | Hz | 0.1 to 100000 | Phase | ° |
| Time | s | 0 to 5 | Voltage | V |

The table shows example combinations of independent (controlled) variables and dependent (measured) variables, and the range over which the scanned variables may be scanned. In an example, a single independent variable of frequency and a single dependent variable of impedance may be used to generate both the first and the second data series. Each electrochemical transducer in the array of wells is interrogated, and the impedance is regularly measured with a signal frequency of 0.1 Hz to generate the first data series. Similarly, the impedance is regularly measured with a signal frequency of 1 Hz to generate the second data series.

While the method 200 refers only to a first and a second data series, this is done to aid quick understanding of the invention and to enable brevity of description. The disclosed method may comprise repeatedly scanning, for example, the independent variable through a plurality of values and recording the dependent variable at each value, and in this way generating a plurality of data series. Extending the above example to an implementation which uses seven data series x₁-x₇, an implementation of the present method may repeatedly measure:
x1=Impedance at 0.1 Hz
x2=Impedance at 1 Hz
x3=Impedance at 10 Hz
x4=Impedance at 100 Hz
x5=Impedance at 1e3 Hz
x6=Impedance at 1e4 Hz
x7=Impedance at 1e5 Hz.

At a high level, the interrogation of the transducers may involve the sending of control signals, from the control unit 120, to the plurality of electrochemical transducers comprised on the cartridge 110. These control signals enable the control of the independent variable(s). Measurement signals are then returned to the control unit 120 which are indicative of the measured dependent variable(s). The interrogation/measurement proceeds at every electrochemical cell present on the system's cartridge 110 in an independent manner. The measurements may run, for example, sequentially in every independent cell in the same order.

At block 230, the method comprises determining, based on the first and second data series from each well, whether there has been microbial growth in any of the wells of the array of wells. The determination may involve making use of the pro-processing, machine learning/numerical models, models, and expert system techniques described herein.

In addition to enabling determination of whether there has been microbial growth in a cell, if there has been microbial growth, the presently disclosed methods also enable a determination of whether a pathogen present in the sample is susceptible or resistant to a particular antimicrobial agent. Further, if the cartridge is prepared accordingly, the present methods enable a determination, for each of a plurality of antimicrobial agents, of whether the pathogen is susceptible to that antimicrobial agent, whether the pathogen is resistant to the antimicrobial agent, and even which of the plurality of antimicrobial agents would be most effective in treating the pathogen.

To enable this functionality, the wells of the array of wells are prepared such that they fall into one of various subsets: a control subset in which the sample will be placed but in which no antimicrobial agent is placed; a first subset in which the sample will be placed and which comprises a first antimicrobial agent; a second subset in which the sample will be placed and in which a second antimicrobial agent will be placed; and so on depending on the number of antimicrobial agents to be tested. The number of wells in each subset may be any number of wells, and there may be a single well in each subset. Optionally, a baseline subset may be included, in which there is no antimicrobial agent and which does not contain the sample. This baseline subset enables pre-processing methods that will be discussed in detail later.

More generally, the system 100 interrogates the following states (or wells) of sample on the cartridge:
Uninhibited sample (sample, gel). This may be described as a positive control.
Inhibited sample(s) (sample, gel, AB).

Optionally, the system may also interrogate the following states (or wells) of sample on the cartridge:
Baseline (sample, gel, no AB added, filtered to exclude or severely reduce bacterial count)
Negative control (sample, gel, bactericidal or bacteriostatic agent)

Here, AB refers to antibiotic agent, but any type of antimicrobial agent can be used. When the cartridge is prepared in this way and a sample potentially containing a pathogen is introduced to the array of wells, then repeated interrogation of the wells as described with respect to blocks 210 and 220 generates a plurality of data series for each well. A subset of those data series are associated with the control subset of wells, a subset of those data series are associated with the first subset of wells, a subset of those data series are associated with the second subset of wells, and so on. By processing these data series in the manner described herein, and in particular by determining a growth factor and/or a ‘time-to-positive’ factor for each well, it is possible to enable several useful determinations. For example, is may be determined which of the antimicrobial agents is most suitable for treating the pathogen. It may be determined that the pathogen is resistant to one or more of the antimicrobial agents. Or, a “no growth” result may be returned, which suggests that there is no pathogen present in the sample.

Methods of the present disclosure will now be described under the following sections:
1. Signal processing: to provide clean signal for the models (e.g. smoothing, normalising, etc.)
2. Model(s): predict states based on input data (e.g. numerical fitting or condition checking); Model training and development, and model application;
3. Growth factor evaluation/expert system: interprets outputs from the models (e.g. voting system, rule-based system, etc.)
4. Display of results

### Signal (Pre-)Processing

There are several pre-processing methods, which provide a framework to standardise experimental output. The objective of the pre-processing step is to provide data in a form, which makes comparison between different experiments, instruments, samples, etc. possible, without necessitating a re-calibration step to ensure expected outcomes.

The pre-processing steps are not essential, but are advantageous, and may include:
Normalisation of each trace by division of the entire trace by first measurement value (time t₀=0 s) (see Equation 1)
Subtraction of a mean baseline trace (average of all present baseline readings present on the cartridge from all other well types)

Each measured variable is considered independently for the purpose of pre-processing. E.g. impedance traces at frequency 100 kHz are analysed separately from frequency traces at 10 kHz, etc.

$${x}_{t} = \frac{{x}_{t = i}}{{x}_{t = 0}} \qquad Equation 1$$

where t=i is a timepoint at time ‘i’, t=0 is a first data point acquired within an experiment. Accordingly, each data series associated with a particular well may be pre-processed by dividing the entire data series by the first measurement of that data series as part of a normalisation process.

In an implementation in which the baseline subtraction pre-processing step is used, the data series produced by the baseline subset of wells, which may be a single baseline well in which the sample and growth gel is present but there is no antimicrobial agent (e.g. no antibiotic agent). This process reduces noise and improves the accuracy of the results.

### Model(s)

Numerical models enable automated analysis of the transducer outputs to enable meaningful results to be generated by the system 100.

In most implementations, an aim of the system is to provide a decision support tool for the healthcare professional (HCP), informing about clinically relevant states:
Is bacterial matter growing in the sample?
Is the bacterial matter affected (killed) by the on-board AB?

Further goals of the system may include returning a corresponding AST result: ‘S’, ‘R’ or ‘I’ (susceptible, resistant, intermediate).

The results are generated by an on-board algorithm interrogating the traces derived from electrochemical methods, treated by pre-processing steps described above, and analysed using one or more numerical models. Finally, an expert system is tasked to compile the results from the models into a final result shared with the operator (e.g. HCP).

The numerical models used to generate a calibration scheme for the system are of multivariate and/or multivariable nature. As such many techniques may be applied to derive a static calibration model, such as PCR, PLS, GLM, etc.

In an example, a generalised linear model (GLM) approach is used, using multiple variables. for example, the impedance and derivative at selected frequencies may be used. This results in an equation structure of the following form (see Equation 2):

$$y = {ax}_{1} + {bx}_{2} + {cx}_{3} + {dx}_{1} {x}_{2} + {ex}_{1} {x}_{3} + {fx}_{2} {x}_{3} \qquad Equation 2$$

where:
   ‘a’, ‘b’, ‘c’, ‘d’, ‘e’ and ‘f’ are parameters derived during fitting of the model
   x₁ to x₃ are the measured (dependent) variables included in the model (may be expanded to accommodate more variables)
   ‘y’ is a growth factor indicative of the bacterial concentration in a particular well, which in turn is indicative of a cell count in the well.

### Model Training and Development

The development process involves laboratory and clinical studies, to gather information and training data in order to train the model and derive the final configuration of the system. The key questions being answered by laboratory studies are:
Type of AB capable of being run by the system
Expected LoD for each antibiotic as applied to the seeding rate of the bacteria present
Breakpoint concentration for every AB
Expected average ‘time-to-result’ (TTR)
Applicability for specific bacteria
AB stability
Impact of interferents.

Using the GLM approach described above, the general aim is to build an equation (see equation 2 above) which is able to predict a growth factor value (e.g. bacterial concentration) based on a number of measured variables. To construct the equation, several parameters are derived during fitting of the equation.

In an example, gathering the training data for model fitting makes use of traditional cell-counting techniques. Samples of the type that may be encountered during use of the method may be used in order to train or fit the model, for example urine samples from patients with a confirmed UTI. A plurality of samples containing one or more microbes are prepared. Samples with varying amounts of microbial growth (i.e. with varying cell counts) are prepared, for example by introducing microbes to a gel and leaving these prepared samples for different amounts of time. Optionally, different antimicrobial agents may be introduced to some of the samples, and optionally samples are gathered using a variety of different microbes.

Next, a cell-count or bacterial concentration is derived for each sample. In an example, agar diffusion methods such as disk diffusion methods may be used to derive a cell count. Researchers may use a counting chamber such as a haemocytometer to derive a cell-count from each sample. Alternative approaches include flow cytometry. The cell count and cell concentration are both examples of a ‘growth factor’, i.e. a factor which is indicative of the degree of microbial growth which has occurred in each well.

In a preferred implementation, training data stems from experiments conducted in the lab using spiked healthy volunteer urine as well as urine obtained directly from UTI patients. The labelling process involves running routine microbiology methods (VITEK-2 instrument) to identify pathogen (known for spiked samples) and obtain a cell count (by utilising automated cell counting microscope/camera equipment). In addition, an AST result may be identified to give a reference AST outcome.

In addition to recording a cell count, or cell concentration, value for each sample, the electrochemical properties of the samples are investigated in order to produce training data to train one or more numeral models. This is done by interrogating the samples using electrochemical transducers in much the same way as described above in relation to method 200. In an example, system 100 may be used to collect this information. Samples with a known cell count may be inserted into the plurality of wells 111a-g in the cartridge 110. The measurable variables to be included in the numerical model, for example the GLM model, are then measured. Using the GLM example, referring to equation 2 above, and the dependent variables x₁-xₙ are measured. In the example with seven dependent variables mentioned above, for example, the impedance of each sample is measured at 0.1 Hz; 1 Hz; 10 Hz; 100 Hz; 1e3 Hz; 1e4 Hz; 1e5 Hz.

The model training/fitting stage may comprise performing method steps 210 and 220 from FIG. 2, including any of the steps mentioned above with respect to FIG. 2. In other words, the training process may comprise inserting a cartridge 110 comprising wells housing one or more samples with a known cell count into the cartridge-receiving machine 120 (or control unit). Then, for each well of the array of wells, a scan is performed over a first scan variable to generate a first data series, and a second scan is performed over either the same (first), or a second, scan variable to generate a second data series. In this way, training data is collected from samples with a known cell count. This training data can be used to train a numerical model such that, when presented with a corresponding first and second data series for a sample with an unknown cell count, the unknown cell count can be estimated (or ‘determined’).

### Model Application

Once the one or more numerical models have been trained, the model can be used to predict/determine a cell count for a new sample. A sample is introduced to the cartridge and the electrochemical transducers are interrogated. A plurality of data series is produced for each well. In the example with 7 dependent variables x₁-x₇, the impedance is measured in each well while the independent variable frequency is scanned through each of the 7 different frequency values. This process is repeated continuously. As data is collected for each well over time, the one or more trained numerical models can be used in real time to generate an estimate of microbial growth for each well.

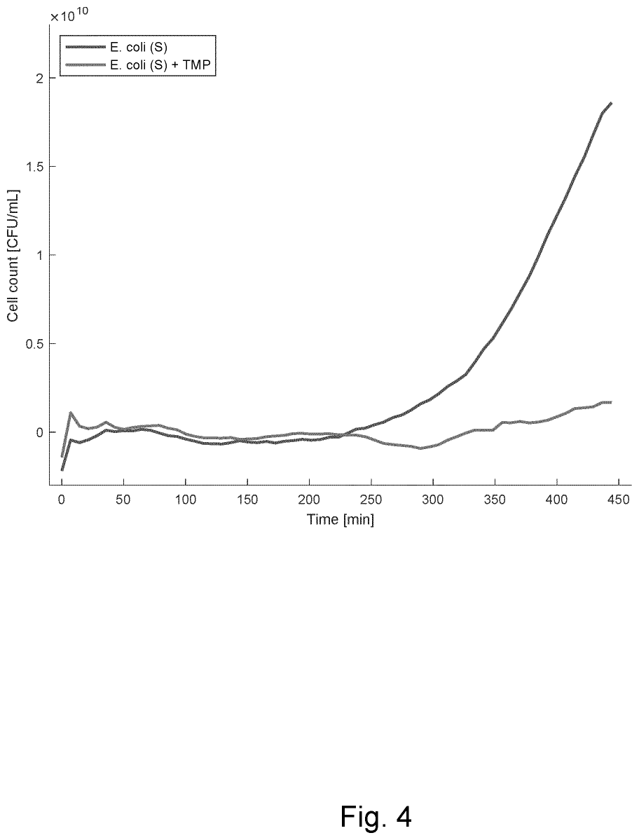

FIG. 4 is a graph which depicts an example output of a trained numerical model. On the y-axis is growth factor, in this case cell count measured in CFU/mL and with a scale of ×10¹⁰. The x-axis depicts time in minutes. The time begins when a cartridge containing a sample is inserted into the control unit 120, or (e.g.) when a user commences the testing process shortly afterwards. In this case, the sample contains Escherichia coli. The graph depicts the outcome of a trained numerical model running on data series associated with a control well, in which there is no antimicrobial agent present (blue line). As can be appreciated, the control growth factor increases quickly over time as microbial growth occurs within the control well. The graph also depicts the outcome of the trained numerical model running on data series associated with another well, which contains trimethoprim (TMP, red line). As can be appreciated, the TMP growth factor levels out over time, suggesting that the presence of TMP is responsible for inhibiting microbial growth within the well.

### Growth Factor Evaluation

The growth factor evaluation process may involve making use of an expert system, which may be referred to as an expert module. The expert system employs an algorithm and/or numerical model in order to generate a useful output on the basis of the growth factors associated with each well.

The evaluation process involves monitoring the control growth factor and comparing it to a minimum or ‘threshold’ growth value. If the control growth factor exceeds the threshold growth value, then it may be determined that there has been microbial growth. In turn, it can be determined that a pathogen is present in the sample. The value of the threshold growth value may be determined and calibrated by reference to laboratory and clinical experiments. This comparison process enables a result of either “growth” or “no growth” to be returned. To give an example with reference to the control growth factor represented in FIG. 4, a suitable minimum growth factor may be 0.25×10¹⁰ CFU/mL, in which case it is possible to return a positive growth result after approximately 300 minutes. This is a simple example to demonstrate the principle and in reality a growth factor can often be determined much sooner than that.

Accordingly, it will be understood that methods and systems of the present disclosure are not only suitable for AST testing, but also for use cases in which it is valuable to identify whether there is a pathogen present in a sample or not, for example for water treatment. To increase accuracy in this use case, the control growth factor (associated with wells which do contain the sample) can be compared to the baseline growth factor (associated with baseline wells which do not contain a sample), and the difference between these monitored growth factors can be monitoring in the manner described generally herein to output a result of either confirmed growth, or confirmed non-growth. In this way, for example, a pathogen in a water supply can be detected.

In a simple example suitable for AST, the evaluation process may additionally involve comparing the growth factors from each inhibited well with the growth factor from the uninhibited well(s) (i.e. the control wells). This comparison is repeated continuously. The difference between the growth factor from each well and the control growth factor may be monitored in real-time. The expert system is configured to determine that the control growth factor associated with the control well(s) has exceeded the growth factor of any of the other wells by a statistically significant amount. For example, the expert system may determine that the difference between the control growth factor and a first growth factor, associated with a first antimicrobial agent, has exceeded a threshold amount. The threshold amount may be a particular predefined amount, or a particular percentage increase. The time at which this statistically significant different/threshold is reached is recorded. This is the ‘time-to-positive’ value for a particular well.

The evaluation process may therefore involve applying a simple logical statement, in which growth factors associated with wells comprising an anti-microbial agent are compared with the control growth factor(s), and time points are identified for an inhibited sample to diminish to (say) 50% of the uninhibited sample. This evaluation process may occur during a particular time period, typically a pre-determined time period. This may be referred to as a threshold time period or a ‘cut-off time’. This threshold time period may be 60 minutes, or 100 minutes, for example.

During the threshold time period, the growth factors for some wells may have fallen under the control growth factor(s) by a statistically significant amount. The antimicrobial agents in these wells are affective at inhibiting growth of the pathogen in the sample. The growth factors for some wells may not have fallen under the control growth factor(s) by a statistically significant amount. The antimicrobial agents in these wells are not affective at inhibiting growth of the pathogen in the sample. The pathogen is resistant to these antimicrobial agents. Alternatively, if bacterial growth has not been detected in the positive control well at a minimum required amount (e.g. cell concentration), a ‘no growth’ result may be returned. This result suggests that there is no pathogen in the sample.

End conditions for the evaluation process include: the threshold time period has expired (i.e. the cut-off time has been reached), a result has been obtained for each inhibited well (wells with antimicrobial agent), or at least one affective antimicrobial agent has been identified.

### Display of Results

Optionally, the results of the evaluation process may be displayed to a user. This allows a clinician to see the results of the evaluation and take appropriate action. Once a result has been obtained for each well (or another end condition has been met) the test is terminated. The data is saved and the result is passed on to the display 1210, i.e. a GUI. The GUI displays the result(s), which may consist of the following items:
Exception handle: error message
No growth detected: message informing the operator that bacterial growth has not been detected in the positive control (sample) well at the minimum required concentration
Growth detected: message informing the operator that bacterial growth has been detected in the positive control (sample) well at the minimum required concentration
Susceptibility: message advising operator that the observed bacterial growth was inhibited by an antibiotic (at the antibiotic's breakpoint concentration)

All outcomes may be presented on the GUI while the test is running. Once the test is concluded, in addition to the GUI, a file is saved and stored on the instrument for future reference. As the measurement process is dynamic, an outcome may occur for any given AB at any stage within the overall test time. Results will be displayed to the operator on the GUI as soon as they are available.

### Summarising Example

To expand upon the present disclosure with an example, the direct output of the trained numerical model may be an estimate of the cell count (dimension “CFU/mL”, i.e. colony forming units per millilitre). This is directly related to a reference cell count. An indirect output of the trained numerical model is the TTR (time to result) once differences between cell count estimates between wells containing antibiotic are visible to those wells where no antibiotic is present (“S” sensitive result). If there are no differences after a set time, a resistant result may be declared (“R”). If no cells count increase is observed within uninhibited wells (no antibiotics present), than no growth result is conveyed.

The numerical model may be a GLM, trained by comparing measured data to a reference vector of cell count estimate and running a minimisation function, which aims at reducing the relative difference between the measured and reference cell count estimates [(CellCount_measured−CellCount_reference_/CellCount_measured].

Training data stems from laboratory observations (experiments conducted in the lab using spiked healthy volunteer urine as well as urine obtained directly from UTI patients. The labelling process involves running routine microbiology methods (VITEK-2 instrument) to identify pathogen (known for spiked samples) and identify the AST result to give a reference AST outcome as well as cell count (by utilising automated cell counting microscope/camera equipment). The data may be gathered from spiked healthy donor urine (may be filtered to remove any unwanted bacteria) and urine from UTI patients.

### Performing AST

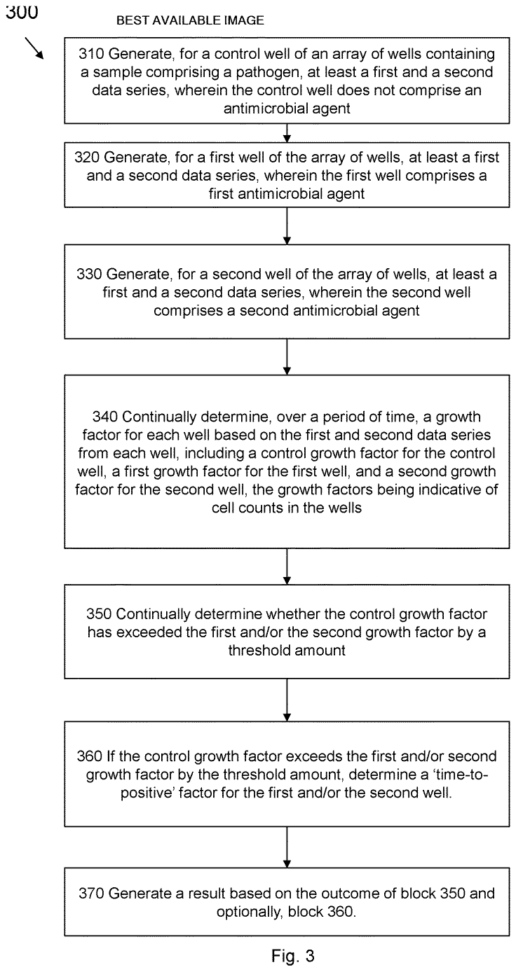

FIG. 3 is a flowchart depicting a method 300. Method 300 is an implementation of the method 200 described above with respect to FIG. 2, which builds upon the method 200 in a manner which renders the method 300 particularly suitable for performing AST. The method is computer-implemented and may be implemented, for example, by one or more processors 1202 on the cartridge-receiving control unit 120.

At blocks 310, 320 and 330, data series are generated for each of a plurality of wells. The wells have been prepared with a sample as set out above. At block 310, a plurality of data series is generated for a control well of the array of wells, comprising at least a first and a second data series. As detailed above, the control well contains a sample which is suspected to comprise a pathogen. The control well does not comprise an antimicrobial agent. At block 320, at least a first and a second data series is generated for a first well of the array of wells, wherein the first well comprises a first antimicrobial agent. At block 330, at least a first and a second data series is generated for a second well of the array of wells, wherein the second well comprises a second antimicrobial agent.

The skilled person will appreciate that while only three wells are explicitly discussed in relation to FIG. 3, the method 300 may be performed for any number of wells each containing different antimicrobial agents. Alternatively, there may be duplication of the same antimicrobial agent across several wells in order to increase the accuracy of the end-result. In this case, an average may be taken of each of the growth factors associated with a particular well subset type, e.g. to generate an average control growth factor, an average growth factor associated with the first antimicrobial agent, and average growth factor associated with a second antimicrobial agent, etc. These average values are then used in the following processing steps as would be understood by the skilled person.

If the aim of the test is to determine whether the potential pathogen is susceptible to a particular (single) antibiotic/antimicrobial agent, then there may only be a single antibiotic/antimicrobial agent present on the cartridge and it will therefore be appreciated that block 330 is optional, and need only be performed if the aim is to perform AST for two different antibiotic/antimicrobial agents.

At block 340, it is continually determined, over a period of time, a growth factor for each well based on the first and second data series from each well. The period of time may also be referred to as a threshold period of time or a cut-off period of time as described above. At this block, a growth factor is continuously generated, using one or more numerical models, for each well being tested. The calculated growth factors will typically include a control growth factor for the control well, a first growth factor for the first well, and a second growth factor for the second well. The growth factors are indicative of cell counts in the wells, and may for example be an estimate of cell count, or of cell concentration in the well. As described above, block 340 may additionally comprise adjusting the estimated growth factors for each well based on the results from the baseline well, in which there is no antimicrobial agent or sample.

At block 350, it is determined whether the control growth factor has exceeded the first and/or the second growth factor by a threshold amount. This determination is performed continually to enable a real-time identification of a result, and which optionally can be relayed to a user immediately. In an example, the difference between the control growth factor and the first growth factor (and the difference between the control growth factor and the second growth factor) is monitored in real-time. This is part of the evaluation process discussed above. If the growth factor for a particular well diminishes compared to the control growth factor by the threshold amount, it implies that the antimicrobial agent in that well is effective against the pathogen comprised within the sample.

At block 360, useful outputs are generated based on the continuous determination/monitoring process performed at block 350. If the control growth factor exceeds the first and/or second growth factor, determine a ‘time-to-positive’ factor for the first and/or the second well. The ‘time-to-positive’ factor is indicative of the time taken for the control growth factor to exceed the first and/or the second growth factor by the threshold amount. The ‘time-to-positive’ factor is determined based on the time at which the growth factor in a well meets the threshold criterion. For example, if the pathogen is susceptible to the first antimicrobial agent, a first ‘time-to-positive’ factor is determined which is indicative of the time taken for the control growth factor to become greater than the first growth factor by the threshold amount.

At block 370, a result is generated based on the outcome of blocks 350 and 360. This result may comprise, determining, based on the first and second ‘time-to-positive’ factors, which of the first and second antimicrobial agents is more suitable for treating the pathogen.

The results may be any of the results discussed herein. For example, if the control growth factor exceeds a minimum for the control growth factor, then it is determined that there has been microbial growth. If the minimum control growth factor has been exceeded within the period of time, then it is determined that there has not been microbial growth, which may in turn suggest that the sample did not contain a pathogen.

For each of the first and second antimicrobial agent, if the control growth factor did exceed the first and/or the second growth factor by the threshold amount at block 350, then a ‘susceptible’ result is generated. This indicates that the pathogen is susceptible to the first and/or the second antimicrobial agent. A list of which antimicrobial agents were found to be effective may be generated, and optionally may be displayed to the user and/or saved in an appropriate file format.

if a ‘time-to-‘positive’ factor has been established for each of the first and the second antimicrobial agent at block 360, then it can be determined, based on the first and second ‘time-to-positive’ factor, which of the first and second antimicrobial agents is most suitable for treating the pathogen. The antimicrobial agent associated with the shortest ‘time-to-positive’ may be determined to be the most effective. This method can be extended to any number of antimicrobial agents and accordingly it will be appreciated that the presently disclosed method enables rapid and effective AST testing for each of a potentially large number of antimicrobial agents.

If the control growth factor has not increased above the first (and/or the second) growth factor within the time period, and it has also been determined that microbial growth is present in the control well, then it may be determined that the pathogen is resistant to the first (and/or the second) antimicrobial agent.

### Example Using an Optimized GLM

The following is an example using a generalised linear model (GLM) based on a single main (dependent) variable: impedance. The main variable is expanded into a multivariate measurement by scanning impedance through frequencies of:
x1=Impedance at 0.1 Hz
x2=Impedance at 1 Hz
x3=Impedance at 10 Hz
x4=Impedance at 100 Hz
x5=Impedance at 1e3 Hz
x6=Impedance at 1e4 Hz
x7=Impedance at 1e5 Hz

The following table (“Table 2”) describes the entries into the GLM equation, derived during a training phase, following the pattern set in “Equation 2”.

TABLE 2
GLM parameters

| | Parameter |
|---|---|
| Term | Value |
| Intercept | 0 |
| x2 | −4.01e10 |
| x6 | 6.568e10 |
| x1:x3 | −5.67e10 |
| x1:x4 | 1.183e11 |
| x1:x5 | −9.48e10 |
| x1:x7 | −3.57e10 |
| x2:x3 | 4.103e10 |
| x2:x4 | −1.05e11 |
| x2:x5 | 1.794e11 |
| x2:x6 | −1.35e11 |
| x2:x7 | 4.918e10 |
| x3:x4 | 5.601e9 |
| x3:x5 | −5.14e10 |
| x4:x5 | 2.973e10 |
| x6:x7 | 2.386e10 |

The terminology “x1:x3” (e.g.) in Table 2 above means “value of measure x1 multiplied by value of measure x3”. This is conducted for every time point.

Using the derived parameters from Table 2, the generic Equation 2 is adapted to describe the example model in its entirety. The model equation is presented in full in Equation 3.

$$y = 0 + 4.01 e 10 \times {x}_{2} + 6.568 e 10 \times {x}_{6} - 5.67 e 10 \times {x}_{1} \times {x}_{3} + 1.183 e 11 \times {x}_{1} \times {x}_{4} - 9.48 e 10 \times {x}_{1} {x}_{5} - 3.57 e 10 \times {x}_{1} \times {x}_{7} + 4.103 e 10 \times {x}_{2} \times {x}_{3} - 1.05 e 11 \times {x}_{2} \times {x}_{4} + 1.79 e 11 \times {x}_{2} \times {x}_{5} - 1.35 e 11 \times {x}_{2} \times {x}_{6} + 4.918 e 10 \times {x}_{2} \times {x}_{7} + 5.601 e 9 \times {x}_{3} \times {x}_{4} - 5.14 e 10 \times {x}_{3} \times {x}_{5} + 2.973 e 10 \times {x}_{4} \times {x}_{5} + 2.386 e 10 \times {x}_{6} \times {x}_{7} \qquad Equation 3$$

The intercept of ‘0’ can of course be omitted and is included in the Equation 3 only to provide direct relation between Table 2 and Equation 3.

The skilled person will understand that, though the above example has been described with respect to determining impedance at each of seven frequencies, any number of frequency values can be monitored, within the range of the potentiometer (see table 1). Also, any of a plurality of dependent/independent variables can be used to construct a working GLM (See table 1). For example, the method may comprise detecting both impedance and capacitance at each of the seven frequency values above, or just capacitance. The method would proceed to constructing a trained GLM in a manner similar to that described above.

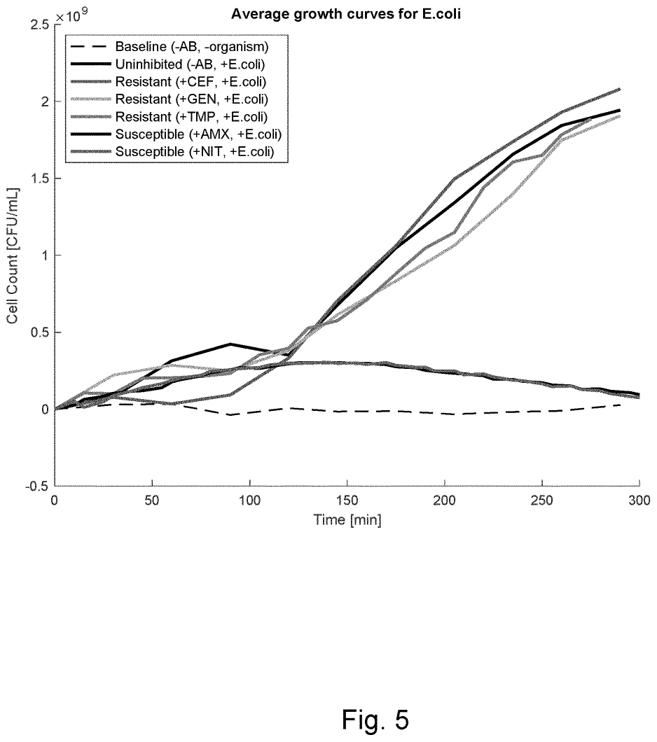

FIG. 5 depicts a graph taking the same form as that depicted in FIG. 4, and presents relative the cell concentration response as returned by the model. The graph depicts the average growth curve prediction for each of a number of different well subset types for a time window 0-300 min. In particular, the graph depicts the behaviour over time of an estimated cell concentration for each of the following well subsets:
baseline (no antimicrobial agent, no sample), dashed line
control/uninhibited wells (no antimicrobial agent, sample containing E. coli), solid, thick black line
first subset of wells (Cefadroxil, CEF, sample containing E. coli), solid, purple line
second subset of wells (Gentamicin, CEF, sample containing E. coli), solid, yellow line
third subset of wells (Trimethoprim, TMP, sample containing E. coli), solid, red line
fourth subset of wells (Amoxicillin, AMX, sample containing E. coli), solid, thinner black line
fifth subset of wells (Nitrofurantoin, NIT, sample containing E. coli), solid, blue line

There are multiple wells in each subset which are prepared in the same way. The graph depicts the mean average growth factor for each subset. For example, the control growth factor depicted on the graph is the mean average of the control growth factor associated with each control well.

The graph also indicates the result obtained for each subset/category of well being tested: E. coli was found to be resistant to the antimicrobial agent in each of the first, second, and third subset of wells, while E. coli was found to be susceptible to the antimicrobial agents in the fourth and fifth subset of wells.

The model has been trained to cater for a long duration of experimental interrogation of an UTI sample. This is particularly relevant to avoid misclassification of a “R” result (organism is resistant to the antibiotic). It is also possible to train a different model, which may return an “S” (organism is susceptible to the antibiotic) result sooner.

The model was optimised using the following steps:
“fitglm” in MATLAB was used to establish a preliminary model based on all Impedance variables.
Non-significant estimates of parameter terms were rejected (pValue>0.05)
Model was re-optimised using the “fminunc” function with the retained terms from Table 2.

The model fitness function is based on the minimisation of prediction error in relative terms (i.e. calculated cell count output subtracted from the reference cell count divided by the calculated value).

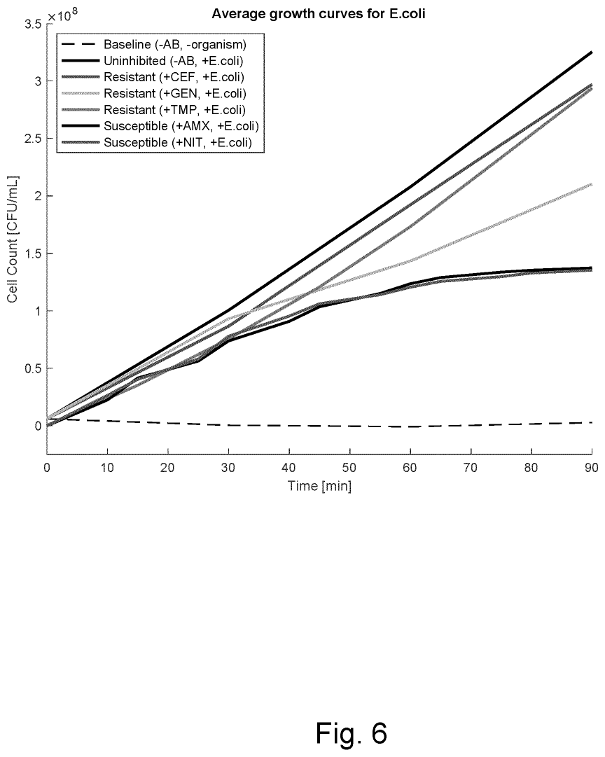

In this example, the model is tuned to a specific time range. The example model (Equation 3) is tuned to operate in a time frame of 0-300 min. In addition, or alternatively, a more aggressive model (same structure with different coefficients) can be utilised to maximise the quality of early predictions. An example of the output of such a model is depicted in FIG. 6.

FIG. 6 is a graph taking the same form as FIGS. 4 and 5, and depicts the estimated cell concentration values for the same well subsets described above with reference to FIG. 5. However, the model used to generate the results depicted in FIG. 6 is more ‘aggressive’, and depicts an operational time between 0-90 min.

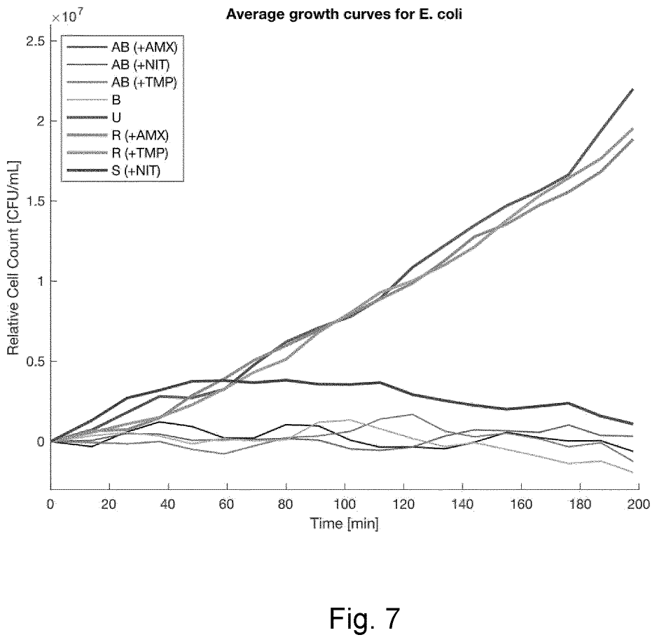

FIG. 7 is a graph taking the same form as FIGS. 4 and 5, and depicts estimated cell concentration values in another example of a GLM similar to that described above. The graph depicts the average growth curve prediction for each of a number of different well subset types for a time window 0-200 min.

In particular, the graph depicts the behaviour over time of an estimated cell concentration for each of the following well subsets:
baseline (no antimicrobial agent, no sample), narrow yellow line
control/uninhibited wells (no antimicrobial agent, sample containing E. coli), thick purple line
first subset of control wells (Amoxicillin, AMX, no sample), narrow black line
second subset of control wells (Nitrofurantoin, NIT, no sample), narrow blue line
third subset of control wells (Trimethoprim, TMP, no sample), narrow orange line
first subset of sample wells (Amoxicillin, AMX, sample containing E. coli), thick green line
second subset of sample wells (Trimethoprim, TMP, sample containing E. coli), thick blue line
third subset of sample wells (Nitrofurantoin, NIT, sample containing E. coli), thick red line

There are multiple wells in each subset which are prepared in the same way. The graph depicts the mean average growth factor for each subset. For example, the control growth factor depicted on the graph is the mean average of the control growth factor associated with each control well.

The graph also indicates the result obtained for each subset/category of well being tested: E. coli was found to be resistant to the antimicrobial agent in each of the first and second subset of sample wells (containing AMX and TMP, respectively), while E. coli was found to be susceptible to the antimicrobial agent in the third subset of sample wells (containing NIT).

### Example Using a Partial Least Squares (PLS) Model

A partial least squares (PLS) model may also be computed. In one example, in addition to the impedance variables as described above (e.g., as described above with respect to the GLM), the model may use current values as input parameters. However, it will be appreciated that any one or more variables as described herein may be used.

Table 3 shows a table of the entries into a PLS model, in which terms x1 to x7 are impedance variables at different frequencies as described above with respect to the GLM, and terms x8 to x10 are measurements of current. In particular, terms x8 to x10 are current measurements taken at times of:
x8=current at sweep time 0.5 s
x9=current at sweep time 2.5 s
x10=current at sweep time 4.5 s

TABLE 3
PLS parameters

| | | Parameter |
|---|---|---|
| Term | Parameter | Value |
| Intercept (x0) | pₓ₀ | 0.0529e9 |
| x1 | pₓ₁ | 0.0156e9 |
| x2 | pₓ₂ | −0.5832e9 |
| x3 | pₓ₃ | 2.6697e9 |
| x4 | pₓ₄ | −5.3870e9 |
| x5 | pₓ₅ | 6.0631.e9 |
| x6 | pₓ₆ | 0.0443e9 |
| x7 | pₓ₇ | 2.0522e9 |
| x8 | pₓ₈ | 0.0053e9 |
| x9 | pₓ₉ | −0.0035e9 |
| x10 | pₓ₁₀ | 0.0365e9 |

The computation of the PLS model is conducted as a matrix multiplication. Here, the observations (each corresponding to a sample) are arranged in rows, with each column describing a single variable. That is, variable ‘x1’ is in column 1, variable ‘x2’ is in column 2, etc.

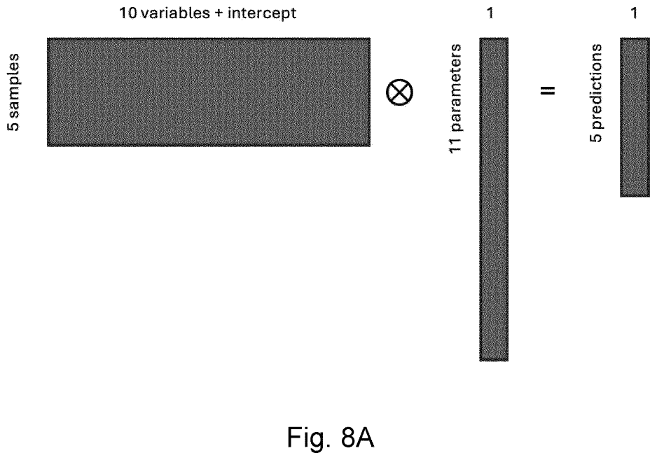

FIG. 8A shows a schematic of the matrix multiplication and FIG. 8B shows a detail matrix multiplication based on 5 samples and the variables shown in Table 3.

As shown in FIG. 8B in order to obtain a prediction for sample 1 the following equations must be calculated:

$${s}_{1} = s {1}_{x 0} \times {p}_{x 0} + s {1}_{x 1} \times {p}_{x 1} + s {1}_{x 2} \times {p}_{x 2} + s {1}_{x 3} \times {p}_{x 3} + s {1}_{x 4} \times {p}_{x 4} + s {1}_{x 5} \times {p}_{x 5} + s {1}_{x 6} \times {p}_{x 6} + s {1}_{x 7} \times {p}_{x 7} + s {1}_{x 8} \times {p}_{x 8} + s {1}_{x 9} \times {p}_{x 9} + s {1}_{x 10} \times {p}_{x 10} \qquad Equation 4$$

Similarly, for sample 2 the following equation must be calculated:

$${s}_{2} = s {2}_{x 0} \times {p}_{x 0} + s {2}_{x 1} \times {p}_{x 1} + s {2}_{x 2} \times {p}_{x 2} + s {2}_{x 3} \times {p}_{x 3} + s {2}_{x 4} \times {p}_{x 4} + s {2}_{x 5} \times {p}_{x 5} + s {2}_{x 6} \times {p}_{x 6} + s {2}_{x 7} \times {p}_{x 7} + s {2}_{x 8} \times {p}_{x 8} + s {2}_{x 9} \times {p}_{x 9} + s {2}_{x 10} \times {p}_{x 10} \qquad Equation 5$$

The remaining sample predictions (i.e., for samples s3 to s5) are calculated in a similar manner. As shown in FIG. 8B this results in a matrix containing predictions for each sample s1 to s5.

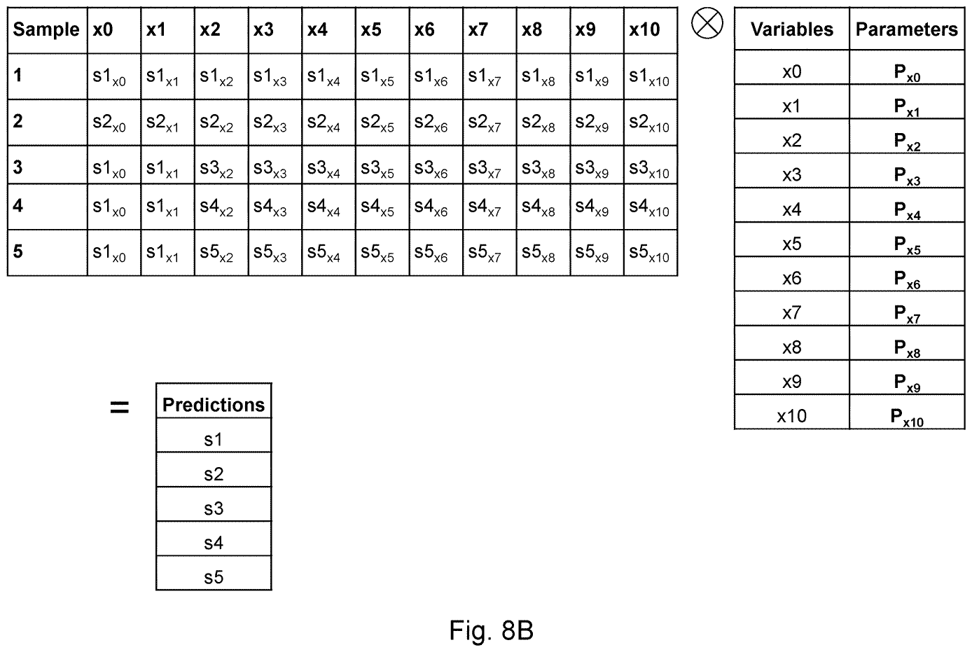

FIG. 9 is a graph showing an average curve prediction based on a PLS model. FIG. 8 depicts a graph taking the same form as that depicted in FIG. 4, and presents relative the cell concentration response as returned by the PLS model.

The graph depicts the average growth curve prediction for each of a number of different well subset types for a time window 0-200 min. In particular, the graph depicts the behaviour over time of an estimated cell concentration for each of the following well subsets:
baseline (no antimicrobial agent, no sample), narrow purple line
control/uninhibited wells (no antimicrobial agent, sample containing E. coli), thick green line
first subset of control wells (Amoxicillin, AMX, no sample), narrow blue line
second subset of control wells (Nitrofurantoin, NIT, no sample), narrow orange line
third subset of control wells (Trimethoprim, TMP, no sample), narrow yellow line
first subset of sample wells (Amoxicillin, AMX, sample containing E. coli), thick light blue line
second subset of sample wells (Trimethoprim, TMP, sample containing E. coli), thick red line
third subset of sample wells (Nitrofurantoin, NIT, sample containing E. coli), thick dark blue line

There are multiple wells in each subset which are prepared in the same way. The graph depicts the mean average growth factor for each subset. For example, the control growth factor depicted on the graph is the mean average of the control growth factor associated with each control well.

The graph also indicates the result obtained for each subset/category of well being tested: E. coli was found to be resistant to the antimicrobial agent in each of the first and second subset of sample wells (containing AMX and TMP, respectively), while E. coli was found to be susceptible to the antimicrobial agent in the third subset of sample wells (containing NIT).

### Principal Component Regression (PCR)

A PCR model may also be computed. The PCR method may use the same model input variables as the PLS method described above. PCR also uses the same matrix multiplication principles as described above to obtain predictions from samples.

Table 4 shows a table of the entries into a PCR model, which are the same as the entries for the PLS model as described above. That is, terms x1 to x7 are impedance variables at different frequencies as described above with respect to the GLM, and terms x8 to x10 are measurements of current.

TABLE 4
PCR parameters

| | | Parameter |
|---|---|---|
| Term | Parameter | Value |
| Intercept (x0) | pₓ₀ | 0.0688e9 |
| x1 | pₓ₁ | −0.4283e9 |
| x2 | pₓ₂ | 0.2741e9 |
| x3 | pₓ₃ | 1.2192e9 |
| x4 | pₓ₄ | −0.5398e9 |
| x5 | pₓ₅ | 3.1775e9 |
| x6 | pₓ₆ | −4.1974e9 |
| x7 | pₓ₇ | −1.9546e9 |
| x8 | pₓ₈ | −0.0787e9 |
| x9 | pₓ₉ | 2.5319e9 |
| x10 | pₓ₁₀ | 0.0053e9 |

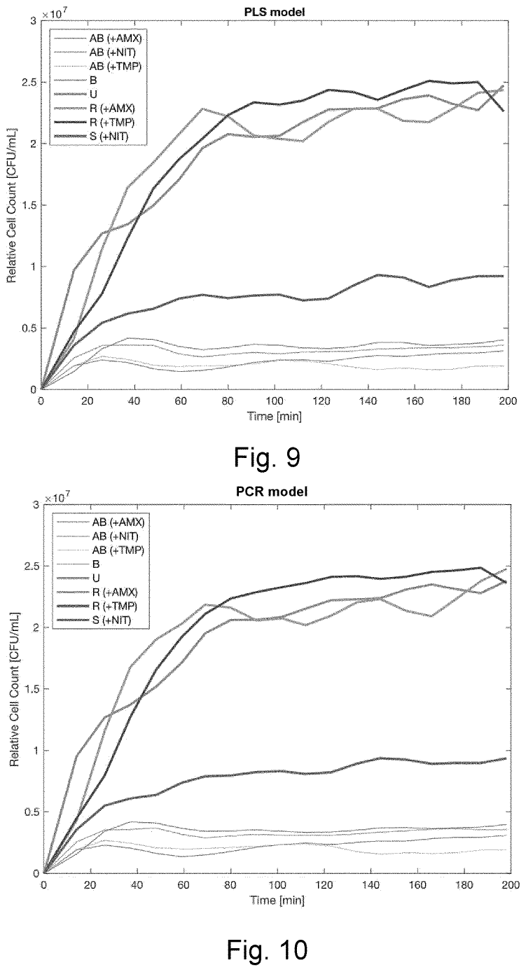

FIG. 10 is a graph showing an average curve prediction based on a PCR model. FIG. 10 depicts a graph taking the same form as that depicted in FIG. 4, and presents relative the cell concentration response as returned by the PCR model.

The graph depicts the average growth curve prediction for each of a number of different well subset types for a time window 0-200 min. In particular, the graph depicts the behaviour over time of an estimated cell concentration for each of the following well subsets:
baseline (no antimicrobial agent, no sample), narrow purple line
control/uninhibited wells (no antimicrobial agent, sample containing E. coli), thick green line
first subset of control wells (Amoxicillin, AMX, no sample), narrow blue line
second subset of control wells (Nitrofurantoin, NIT, no sample), narrow orange line
third subset of control wells (Trimethoprim, TMP, no sample), narrow yellow line
first subset of sample wells (Amoxicillin, AMX, sample containing E. coli), thick light blue line
second subset of sample wells (Trimethoprim, TMP, sample containing E. coli), thick red line
third subset of sample wells (Nitrofurantoin, NIT, sample containing E. coli), thick dark blue line

There are multiple wells in each subset which are prepared in the same way. The graph depicts the mean average growth factor for each subset. For example, the control growth factor depicted on the graph is the mean average of the control growth factor associated with each control well.

The graph also indicates the result obtained for each subset/category of well being tested: E. coli was found to be resistant to the antimicrobial agent in each of the first and second subset of sample wells (containing AMX and TMP, respectively), while E. coli was found to be susceptible to the antimicrobial agent in the third subset of sample wells (containing NIT).

According to methods of the present disclosure, it is possible to use multiple numerical models tuned to different timescales, for example a first model tuned to maximise the quality of early predictions, and a second model tuned to prevent misclassification of an ‘R’ result.

Methods of the present disclosure generate a result based on at least a first and a second data series from each well. This multivariate and multivariable approach generates a wealth of data which can be used to determine whether a particular antibiotic agent is suitable for treating a bacteria comprised within the sample, or which of a number of candidate antibiotics is most suitable for treating the bacteria. It is this multivariate and multivariable approach which allows the time before a final result, whether that be identification of growth or full AST result, to be reduced from 2 days (as in traditional cell growth approaches) to 2 hours, or less.

FIG. 10 depicts a general overview of the presently disclosed methods. At block 1005, raw data is collected via interrogating electrochemical transducers to generate data series associated with a sample. At block 1010, pre-processing is performed, which may comprise baseline subtraction and/or data series normalisation as described above. At blocks 1015 and 1020, standard noise filtering and scaling may be applied to the data series (or ‘traces’). Blocks 1025, 1030 and 1035 describe design features, interpretation of electrochemical method, and feature extraction. At blocks 1040 and 1045, the numerical models are reviewed and the selected model is applied to each channel output (data series from each well) as appropriate). Blocks 1050 and 1055 describe the model output/result evaluation process, which may be performed by an expert system, and the channel behaviour is compared and evaluated. At block 1060, the final result is generated and verified, and at block 1065 the result is output for the user, e.g. via a GUI.

The various methods described above may be implemented by a computer program. The computer program may include computer code arranged to instruct a computer to perform the functions of one or more of the various methods described above. The computer program and/or the code for performing such methods may be provided to an apparatus, such as a computer, on one or more computer readable media or, more generally, a computer program product. The computer readable media may be transitory or non-transitory. The one or more computer readable media could be, for example, an electronic, magnetic, optical, electromagnetic, infrared, or semiconductor system, or a propagation medium for data transmission, for example for downloading the code over the Internet. Alternatively, the one or more computer readable media could take the form of one or more physical computer readable media such as semiconductor or solid state memory, magnetic tape, a removable computer diskette, a random access memory (RAM), a read-only memory (ROM), a rigid magnetic disc, and an optical disk, such as a CD-ROM, CD-R/W or DVD.

In an implementation, the modules, components and other features described herein can be implemented as discrete components or integrated in the functionality of hardware components such as ASICS, FPGAs, DSPs or similar devices.

In addition, the modules and components can be implemented as firmware or functional circuitry within hardware devices. Further, the modules and components can be implemented in any combination of hardware devices and software components, or only in software (e.g., code stored or otherwise embodied in a machine-readable medium or in a transmission medium).

Unless specifically stated otherwise, as apparent from the following discussion, it is appreciated that throughout the description, discussions utilizing terms such as “receiving”, “determining”, “comparing”, “enabling”, “maintaining,” “identifying,” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The approaches described herein may be embodied on a computer-readable medium, which may be a non-transitory computer-readable medium. The computer-readable medium carrying computer-readable instructions arranged for execution upon a processor so as to make the processor carry out any or all of the methods described herein.

The term “computer-readable medium” as used herein refers to any medium that stores data and/or instructions for causing a processor to operate in a specific manner. Such storage medium may comprise non-volatile media and/or volatile media. Non-volatile media may include, for example, optical or magnetic disks. Volatile media may include dynamic memory. Exemplary forms of storage medium include, a floppy disk, a flexible disk, a hard disk, a solid state drive, a magnetic tape, or any other magnetic data storage medium, a CD-ROM, any other optical data storage medium, any physical medium with one or more patterns of holes, a RAM, a PROM, an EPROM, a FLASH-EPROM, NVRAM, and any other memory chip or cartridge.

It is to be understood that the above description is intended to be illustrative, and not restrictive. Many other implementations will be apparent to those of skill in the art upon reading and understanding the above description. Although the present disclosure has been described with reference to specific example implementations, it will be recognized that the disclosure is not limited to the implementations described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

1. A system for identifying microbial growth, the system comprising:
a cartridge for receiving a sample that may comprise a pathogen, the cartridge comprising an array of wells, each well comprising an electrochemical transducer; and
a processor configured to interrogate each of the electrochemical transducers, wherein interrogation of the electrochemical transducers comprises, for each well;
generating a first data series by repeatedly measuring a first dependent variable; and generating a second data series by repeatedly measuring either the first, or a second dependent variable;
wherein the processor is further configured to:
determine, based on the first and second data series from each well,
whether there has been microbial growth in any of the wells.

2. The system of claim 1, wherein each well of a control subset of wells does not comprise an antimicrobial agent, and each well of a first subset of wells comprises a first antimicrobial agent.

3. The system of claim 2, wherein the processor is further configured to determine whether the first antimicrobial agent is suitable for treating the pathogen comprised within the sample.

4. The system of claim 3, wherein each well of a second subset of wells comprises a second antimicrobial agent, and the processor is further configured to determine which of the first and second antimicrobial agent is most suitable for treating the pathogen.

5. The system of claim 1, wherein the sample is a patient sample and comprises at least one of blood, urine, sputum, and cerebrospinal fluid.

6. The system of claim 1, wherein interrogation of the electrochemical transducers comprises, for each well:
generating the first data series by repeatedly measuring the first dependent variable while controlling a first independent variable; and
generating the second data series by repeatedly measuring either the first, or the second dependent variable while controlling either the first, or a second, independent variable.

7. The system of claim 6, wherein the first and second independent variables are selected from voltage, frequency, and time.

8. The system of claim 6, wherein the first and the second dependent variables are selected from current, capacitance, impedance, a first derivative of the impedance, a second derivative of the impedance, phase, and voltage.

9. The system of claim 6, wherein:
generating the first data series comprises measuring the first dependent variable while controlling the first independent variable at a first value;
generating the second data series comprises measuring the first dependent variable while controlling the first independent variable at a second value.

10. The system of claim 6, wherein determining whether there has been microbial growth in any of the wells based on the first and second data series from each well comprises using a trained numerical model.

11. The system of claim 10, wherein the trained numerical model is any of a generalised linear model, partial least squares model, linear discriminant analysis model, or a neural network model.

12. The system of claim 10, wherein the trained numerical model has been trained based on training data, the training data comprising measurements of the first dependent variable and/or the second dependent variable in wells comprising a sample with a known growth factor indicative of cell count.

13. The system of claim 10, the processor being further configured to:
determine, using the numerical model and based on the first and second data series from a control well, a control growth factor indicative of a cell count in the control well;
determine, using the numerical model and based on the first and second data series from a first well, a first growth factor indicative of a cell count in the first well.

14. The system of claim 13, wherein the first well comprises a first antimicrobial agent, and the control well does not comprise an antimicrobial agent.

15. The system of claim 14, wherein the processor is further configured to:
continually determine, over a period of time, the first growth factor indicative of a cell count in the first well comprising the first antimicrobial agent, and the control growth factor indicative of a cell count in the control well which does not comprise an antimicrobial agent;
determine whether the control growth factor has become greater than the first growth factor by a threshold amount, and if the control growth factor has become greater than the first growth factor by the threshold amount, determine that the pathogen is susceptible to the first antimicrobial agent.

16. The system of claim 15, wherein the processor is further configured, based on determining that the control growth factor has not become greater than the first growth factor by the threshold amount within a threshold time period, to determine that the pathogen is resistant to the first antimicrobial agent.

17. The system of claim 15, the processor being further configured to:
determine a first ‘time-to-positive’ factor indicative of the time taken for the control growth factor to become greater than the first growth factor by the threshold amount;
continually determine, over a period of time, using the numerical model and based on a first and second data series from a second well, a second growth factor indicative of a cell count in the second well; wherein the second well comprises a second antimicrobial agent different to the first antimicrobial agent;
determine whether the control growth factor has become greater than the second growth factor by a threshold amount, and determine a second ‘time-to-positive’ factor indicative of the time taken for the control growth factor to become greater than the second growth factor by the threshold amount;
determine, based on the first and second ‘time-to-positive’ factors, which of the first and second antimicrobial agents is more suitable for treating the pathogen.

18. A computer-implemented method suitable for identifying microbial growth, the method for use with a system comprising a cartridge for receiving a sample that may comprise a pathogen, the cartridge comprising an array of wells, each well comprising an electrochemical transducer; the method comprising:
interrogating each of the electrochemical transducers, wherein interrogation of the electrochemical transducers comprises, for each well;
generating a first data series by repeatedly measuring a first dependent variable; and generating a second data series by repeatedly measuring either the first, or a second dependent variable;
wherein the method further comprises determining, based on the first and second data series from each well, whether there has been microbial growth in any of the wells.

19. The method of claim 18, wherein each well of a control subset of wells does not comprise an antimicrobial agent, and each well of a first subset of wells comprises a first antimicrobial agent.

20. The method of claim 19, further comprising determining whether the first antimicrobial agent is suitable for treating the pathogen comprised within the sample.

21. The method of claim 20, wherein each well of a second subset of wells comprises a second antimicrobial agent, and the method further comprises determining which of the first and second antimicrobial agent is most suitable for treating the pathogen.

22. The method of claim 18, wherein the sample is a patient sample and comprises at least one of blood, urine, sputum, and cerebrospinal fluid.

23. The method of claim 18, wherein interrogation of the electrochemical transducers comprises, for each well:
generating the first data series by repeatedly measuring the first dependent variable while controlling a first independent variable; and
generating the second data series by repeatedly measuring either the first, or the second dependent variable while controlling either the first, or a second, independent variable.

24. The method of claim 23, wherein the first and second independent variables are selected from voltage, frequency, and time.

25. The method of claim 18, wherein the first and the second dependent variables are selected from current, capacitance, impedance, a first derivative of the impedance, a second derivative of the impedance, phase, and voltage.

26. The method of claim 23, wherein:
generating the first data series comprises measuring the first dependent variable while controlling the first independent variable at a first value;
generating the second data series comprises measuring the first dependent variable while controlling the first independent variable at a second value.

27. The method of claim 18, wherein determining whether there has been microbial growth in any of the wells based on the first and second data series from each well comprises using a trained numerical model.

28. The method of claim 27, wherein the trained numerical model is any of a generalised linear model, partial least squares model, linear discriminant analysis model, or a neural network model.

29. The method of claim 27, wherein the trained numerical model has been trained based on training data, the training data comprising measurements of the first dependent variable and/or the second dependent variable in wells comprising a sample with a known growth factor indicative of cell count; optionally wherein the method comprises training the model.

30. The method of claim 29, the processor being further configured to:
determine, using the numerical model and based on the first and second data series from a control well, a control growth factor indicative of a cell count in the control well;
determine, using the numerical model and based on the first and second data series from a first well, a first growth factor indicative of a cell count in the first well.

31. The method of claim 30, wherein the first well comprises a first antimicrobial agent, and the control well does not comprise an antimicrobial agent.

32. The method of claim 31, further comprising:
continually determining, over a period of time, the first growth factor indicative of a cell count in the first well comprising the first antimicrobial agent, and the control growth factor indicative of a cell count in the control well which does not comprise an antimicrobial agent;
determining whether the control growth factor has become greater than the first growth factor by a threshold amount, and if the control growth factor has become greater than the first growth factor by the threshold amount, determining that the pathogen is susceptible to the first antimicrobial agent.

33. The method of claim 32, further comprising determining, based on determining that the control growth factor has not become greater than the first growth factor by the threshold amount within a threshold time period, that the pathogen is resistant to the first antimicrobial agent.

34. The method of claim 32, further comprising:
determining a first ‘time-to-positive’ factor indicative of the time taken for the control growth factor to become greater than the first growth factor by the threshold amount;
continually determining, over a period of time, using the numerical model and based on a first and second data series from a second well, a second growth factor indicative of a cell count in the second well; wherein the second well comprises a second antimicrobial agent different to the first antimicrobial agent;
determining whether the control growth factor has become greater than the second growth factor by a threshold amount, and determine a second ‘time-to-positive’ factor indicative of the time taken for the control growth factor to become greater than the second growth factor by the threshold amount;
determining, based on the first and second ‘time-to-positive’ factors, which of the first and second antimicrobial agents is more suitable for treating the pathogen.

35. A computer readable medium comprising computer-executable instructions which, when executed by a processor, cause the processor to perform the method of claim 18.
